(19) United States
(12) Patent Application Publication
(10) Pub. No.: US 2011/0160503 A1
(43) Pub. Date: Jun. 30, 2011

(54) PROCESS FOR PREPARING A SUPPORTED CATALYST BASED ON Ni AND A METAL FROM GROUP IB, FOR THE SELECTIVE HYDROGENATION OF POLYUNSATURATED HYDROCARBONS

(75) Inventors: Lars FISCHER, Vienne (FR); Anne Claire Dubreuil, Lyon (FR); Cecile Thomazeau, Lyon (FR); Layane Deghedi, Lyon (FR); Jean Pierre Candy, Caluire (FR); Jean Marie Basset, Caluire et Cuire (FR); Fabienne Le Peltier, Rueil Malmaison (FR)

(73) Assignee: IFP ENERGIES NOUVELLES, Rueil-Malmaison Cedex (FR)

(21) Appl. No.: 12/856,932

(22) Filed: Aug. 16, 2010

(30) Foreign Application Priority Data
Aug. 17, 2009 (FR) 09/03.987

(51) Int. Cl. C07C 5/00; B01J 23/89; B01J 37/025; B01J 37/16; B01J 21/08; B01J 37/06

(52) U.S. Cl. 585/250; 502/330; 502/243

(57) ABSTRACT

A process is described for preparing a catalyst comprising at least one porous support and at least one metallic phase containing nickel and at least one metal M from group IB in a proportion such that the molar ratio M/Ni is in the range 0.005 to 0.5, said process comprising at least the following steps in succession:
a1) depositing nickel on at least said support in order to obtain a supported nickel-based monometallic catalyst;
b1) depositing, in the presence of at least one reducing gas and in the absence of any aqueous solvent, at least one organometallic compound of at least said metal M onto said monometallic catalyst.

### CROSS-REFERENCE TO RELATED APPLICATION

This application is related to concurrently filed application “Process for Preparing A Ni/Sn Supported Catalyst for the Selective Hydrogenation of Polyunsaturated Hydrocarbons” by Lars Fischer et al., Attorney Docket No. PET-2637, claiming priority of FR 09/03.988 filed Aug. 17, 2009, incorporated by reference herein.

### TECHNICAL FIELD

The present invention relates to the selective hydrogenation of polyunsaturated compounds present in a hydrocarbon feed such as, for example, in steam cracked gasolines. These gasolines contain gum-generating compounds, in particular diolefins and alkenylaromatics, in particular mixed with mono-olefinic and aromatic compounds. In order to be able to upgrade steam cracked gasolines, they must be freed of their diolefins or respectively alkenylaromatic compounds; the diolefins are selectively hydrogenated to mono-olefins and the alkenylaromatics are selectively hydrogenated to aromatics. More precisely, the invention relates to a process for preparing a supported catalyst containing nickel and at least one metal from group IB, for the selective hydrogenation of polyunsaturated compounds present in hydrocarbon cuts.

### PRIOR ART

Selective hydrogenation treatments are generally carried out on metallic catalysts deposited onto an amorphous or crystalline support. The metals used are metals from group VIII and of these, nickel can be pointed out as being in routine use.

However, nickel catalysts are not sufficiently selective as they have a marked tendency to hydrogenate a large part of the mono-olefins contained in the feed, even when the hydrogenations are carried out at low pressure, of the order of 30 to 50 bar, and at low temperature, in the range 50° C. to 180° C. (degrees Celsius).

It is known that improving the selectivity of those catalysts can be achieved by injecting sulphur-containing compounds before bringing the catalyst into contact with the reactive feed in order to obtain a catalyst passivated with sulphur. These compounds may be selected from the following compounds: thiophene, thiophane, alkylmonosulphides such as dimethylsulphide, diethylsulphide, dipropylsulphide or propylmethylsulphide. Such sulphurization is, however, difficult to carry out as it is necessary for the sulphurized compound to be distributed very evenly throughout the catalytic bed in order to achieve a marked effect on selectivity. Furthermore, that procedure is expensive and lengthy, which results in a loss of production.

The intrinsic lack of selectivity of a nickel catalyst is not only manifested as regards mono-olefins, but also as regards aromatics. If the surface of the Ni particles were surface passivated by sulphur-containing organic compounds in a highly controlled manner, a priori onto clearly distinct Ni sites, and in an amount of one atom of S per approximately 4 or 5 surface Ni atoms [T E Fischer, S R Kelemen, J Catal 53, 24 (1978) or GB1565754], hydrogenation of the aromatic rings would be dispensed with and hydrogenation of the olefinic compounds would be greatly slowed. Controlling the deposition of sulphur so closely on an industrial scale as well as controlling the stability of that system under the reaction conditions used for selective hydrogenations (25-30 bar, 50-180° C.) is difficult to achieve, however. Thus, when starting up with a nickel catalyst, even when passivated by a sulphur-containing compound of the type indicated above, it is necessary to use a non-reactive start-up feed, containing neither diolefins nor mono-olefins and very few aromatics. The hydrogenation of the diolefins or possibly the mono-olefins on fresh catalysts which are highly active causes enough heat to be released to raise the temperature of the catalyst to levels well beyond 200° C., which may cause hydrogenation of the aromatics. This latter reaction is even more exothermic and the temperature may exceed 600° C., which results in causing cracking of the hydrocarbons, a reaction which itself is also highly exothermic. In this manner, the temperatures reached during these runaways may exceed the nominal temperatures for steel reactors, meaning replacement not only of the catalyst charge but also of the reactor itself.

Another method for improving catalytic selectivity consists of pre-passivating the Ni catalyst in the oxidized state and in the absence of hydrogen, by impregnation with an organic sulphur compound, for example 2,2-dithio-bis-ethanol (DEODS), as described in patent EP-0 466 567. The polysulphide decomposes in hydrogen in the hydrogenation reactor, simultaneously with the reduction of nickel, and a reactive feed may subsequently be introduced with no danger of reaction runaway. However, given that reduction and passivation with sulphur take place at the same time, it is even more difficult under these conditions to obtain homogeneous passivation of the Ni surface with the desired stoichiometry and only at the desired Ni sites. In order to avoid the presence of non-passivated reduced Ni particles, the polysulphide is introduced in excess and over-passivation with the partial formation of the phase Ni₃S₂ cannot in general be avoided, which significantly reduces the catalytic activity even as regards diolefins (B W Hoffer, R L C Bonne, A D van Langeveld, C Griffiths, C M Lok, J A Moulijn, Fuel 83 (2004), 1-8).

It is also known that bimetallic catalysts may produce gains in selectivity and stability. For selective hydrogenation catalysts, it is known that associations of palladium with a second metal render the catalysts more selective but less active. Such associations have only been proposed for the selective hydrogenation of cuts containing hydrocarbons containing between 2 and 4 carbon atoms, in which usually a first reactor operates with a monometallic palladium catalyst to carry out the major portion of the conversion, and a second reactor containing a bimetallic catalyst completes the conversion in a more selective manner. As an example, U.S. Pat. No. 5,356,851 discloses that it is advantageous to associate a metal from group VIII (preferably palladium) with an element such as indium or gallium for applications for the selective hydrogenation of polyunsaturated compounds. Similarly, Pd—Cu (U.S. Pat. No. 5,464,802), Pd—Ag (U.S. Pat. No. 4,547,600), Pd—Sn and Pd—Pb (JP-59227829) associations or a combination of palladium and an alkali metal (EP-0 722 776) have been identified for their hydrogenation performances. All of these patents are aimed at improving the mono-olefins yield.

Bimetallic nickel-based catalysts are also described in the prior art. Patent GB 1 565 754 describes the introduction of 10 ppm of an element from the platinum group, such as ruthenium, rhodium, palladium, osmium, iridium or platinum, and preferably palladium, into a catalyst for the selective hydrogenation of C3, C4 cuts or gasoline containing 10% of Ni supported on sepiolite. This introduction has the effect of increasing the reducibility of Ni after regeneration, but the subsequent introduction of sulphide compounds is still necessary for passivation. U.S. Pat. No. 5,997,835 describes the introduction of gold into a supported nickel catalyst by an aqueous phase method for methane steam reforming; the effect of introducing the gold is to slow down catalyst deactivation.

Patent FR-2 792 645 describes the preparation, in an aqueous phase at a pH of less than 10, of a bimetallic catalyst associating a metal from group VIII (preferably platinum, palladium and nickel) and a metal preferably selected from germanium, tin, silver and gold, for the selective hydrogenation of polyunsaturated compounds, with the aim of increasing the yield of olefins. U.S. Pat. No. 5,208,405 describes a process for the selective hydrogenation of diolefins containing 4 to 10 carbon atoms; said process minimizes the formation of paraffins as well as the deactivation of the catalyst, which is a supported catalyst based on nickel and silver, preferably prepared by impregnating a nickel compound and a silver compound simultaneously or in succession. The Ag/Ni atomic ratio is in the range 1 to 8.

U.S. Pat. No. 5,948,942 teaches that it is advantageous to associate a completely reduced metal from group VIII with a partially reduced metal from group IB, preferably copper, for the selective and simultaneous hydrogenation of diolefins and nitriles. Those catalysts are preferably prepared by successive impregnation of the various aqueous solutions of metallic salts, with intermediate calcining and activation. U.S. Pat. No. 6,417,419 describes a supported catalyst for the selective hydrogenation of butadiene, based on copper and an activating metal selected from nickel, cobalt, platinum, palladium and manganese in which at least 50% by weight of the metals are distributed in the outer layer of the support to a thickness of 200 microns. The preparation method is impregnation, co-precipitation, co-gelling or ion exchange, preferably impregnation of a solution of salts of these metals. The advantage is to improve the stability of the catalyst.

The aim of the present invention is to provide a catalyst containing a metallic phase based on nickel and at least one metal from group IB, prepared by a novel preparation process, for the selective hydrogenation of polyunsaturated compounds present in a hydrocarbon cut. More precisely, it proposes providing an alternative catalyst to the nickel-based catalysts passivated with sulphur which are known in the prior art.

### SUMMARY AND ADVANTAGE OF THE INVENTION

The present invention provides a process for preparing a catalyst comprising at least one porous support and at least one metallic phase containing nickel and at least one metal M from group IB in a proportion such that the molar ratio M/Ni is in the range 0.005 to 0.5, said process comprising at least the following steps in succession:
a1) depositing nickel on at least said support in order to obtain a supported nickel-based monometallic catalyst; and
b1) depositing, in the presence of at least one reducing gas and in the absence of any aqueous solvent, at least one organometallic compound of at least said metal M onto said monometallic catalyst.

The present invention also pertains to a process for the selective hydrogenation of a feed of polyunsaturated hydrocarbons, said process comprising passing said feed into at least one reaction unit provided with at least one supported catalyst comprising at least one metallic phase containing nickel and at least one metal M from group IB prepared in accordance with the preparation process of the invention.

The catalyst prepared using the process of the invention and used in a process for the selective hydrogenation of polyunsaturated hydrocarbons produces improved catalytic performances in terms of selectivity for mono-unsaturated compounds. Furthermore, the catalyst prepared using the process of the invention and employed in a process for the selective hydrogenation of polyunsaturated hydrocarbons substantially limits or even eliminates the hydrogenation of aromatic rings present in polyunsaturated compounds, such as aromatic, styrene or indene compounds, which means that runaway of the reactions can be avoided and means that the compounds with an aromatic ring issuing from said selective hydrogenation process of the invention can be upgraded in a variety of applications.

### DESCRIPTION OF THE INVENTION

The present invention concerns a process for preparing a catalyst comprising at least one porous support and at least one metallic phase containing nickel and at least one metal M from group IB in a proportion such that the Molar ratio M/Ni is in the range 0.005 to 0.5, said process comprising at least the following steps in succession:
a1) depositing nickel on at least said support in order to obtain a supported nickel-based monometallic catalyst; and
b1) depositing, in the presence of at least one reducing gas and in the absence of any aqueous solvent, at least one organometallic compound of at least said metal M onto said monometallic catalyst.

In accordance with the invention, the catalyst prepared using the process of the invention comprises a metallic phase containing nickel and at least one metal M from group IB selected from copper, silver and gold, said metals being deposited on a porous support. The nickel content in said catalyst is advantageously in the range 1% by weight to 50% by weight, preferably in the range 5% by weight to 40% by weight and more preferably in the range 8% by weight to 30% by weight with respect to the catalyst mass. The quantity of metal M from group IB varies depending on the nature of said metal: it is advantageously in the range 0.005% by weight to 30% by weight of the catalyst mass when M is copper and in the range 0.01% by weight to 50% by weight of the mass of said catalyst when M is silver or gold. The molar ratio M/Ni is generally in the range 0.005 to 0.5, preferably in the range 0.01 to 0.5, and more preferably in the range 0.03 to 0.3. Said catalyst prepared in accordance with the process of the invention may comprise one or more metal(s) M from group IB. Highly preferably, said metal M from group IB is gold.

The porous support present in the catalyst prepared using the process of the invention generally comprises at least one refractory oxide which is advantageously selected from oxides of metals from groups 2, 3, 4, 13 and 14 of the new periodic table of the elements such as, for example, oxides of magnesium, aluminium, silicon, titanium, zirconium or thorium, alone or as a mixture or mixed with other oxides of metals from the periodic table of the elements. Coal may also be used. The preferred support is selected from aluminas, silicas and silica-aluminas, and more preferably it is an alumina or a silica. The pore volume of the support is generally in the range 0.1 cm³/g to 1.5 cm³/g, preferably in the range 0.5 cm³/g to 1 cm³/g. The specific surface area of the support is generally in the range 10 m²/g to 250 m²/g, preferably in the range 30 m²/g to 200 m²/g and more preferably in the range 40 m²/g to 180 m²/g. Said porous support is advantageously in the form of beads, extrudates, pellets or irregular and non-spherical agglomerates the specific shape of which may result from a crushing step. Highly advantageously, said support is in the form of beads or extrudates.

In accordance with step a1) of the preparation process of the invention, nickel is deposited on the porous support. The nickel can be deposited onto said support using any method which is known to the skilled person. As an example, the deposit is produced by impregnation, consisting of bringing said porous support into contact with at least one aqueous or organic solution of at least one nickel compound or with a suspension of at least one organic or inorganic nickel compound, or deposition may be accomplished using deposition-precipitation methods which are well known to the skilled person [J W Geus, Preparation of Catalysts III, in G Poncelet, P Grange, P A Jacobs (Eds), Elsevier, Amsterdam 1983, 1]. Deposition of the nickel onto the support is optionally followed by one or more washes and/or optionally by evaporating off the solvent. Advantageously, deposition of the nickel onto the porous support is followed by one or more heat or chemical treatments resulting in a monometallic supported catalyst based on nickel mainly in the oxide state or mainly in the metallic state. Said step a1) results in the preparation of a supported nickel-based monometallic catalyst.

In accordance with step b1) of the preparation process of the invention, at least one step is then carried out in the presence of at least one reducing gas and in the absence of any aqueous solvent, for depositing at least one organometallic compound of at least said metal M onto said monometallic Ni-based catalyst. The reducing gas is preferably hydrogen. Said step b1) is carried out at a temperature in the range 10° C. to 100° C., preferably in the range 20° C. to 50° C., and for a period in the range 10 minutes to 24 hours. In accordance with a preferred implementation, said step b1) is carried out in the gas phase. In accordance with a still more preferred implementation, said step b1) is carried out in the liquid phase in an organic solvent, for example heptane or toluene.

The metal(s) M from group IB, preferably gold, is(are) introduced in the form of an organometallic compound of said metal(s). Said organometallic compound of at least said metal M employed for carrying out said step b1) comprises at least one carbon-metal M bond (C-M bond), preferably a carbon-gold bond.

Any organometallic compound of gold comprising at least one C—Au bond is suitable for carrying out said step b1). Advantageously, gold cyanide [AuCN], dimethyl gold acetyl acetonate [Au(CH₃)₂(acac)], dimethyl gold iodide dimer [((CH₃)₂AuI)₂], dimethyl gold carboxylate [(CH₃)₂Au(COOR), where R=CH₃ or tertisobutyl], triphenylphosphine gold chloride [Ph₃PAuCl], dimethyl gold oxinate [(CH₃)₂AuL where L=8-quinolenol] are used. Preferably, the organometallic compound of gold is selected from gold cyanide [AuCN], gold acetyl acetonate [Au(CH₃)₂(acac)] and dimethyl gold iodide dimer [((CH₃)₂AuI)₂]; more preferably, it is gold cyanide [AuCN].

Any organometallic compound of silver comprising at least one C—Ag bond is suitable for carrying out said step b1). Advantageously, silver cyanide [AgCN], silver acetyl acetonate [Ag(acac)], N,N′-diisopropylacetamidinato silver [(Ag(iPr-Me-AMD))ₓ, where (x=2,3)], trans-bis(trimethylsilyl)ethene(1,1,1,5,5,5-hexafluoro-2,4-pentanedionato) silver, silver ethylhexanoate [AgOOCCH(C₂H₅)C₄H₉)] are used. Preferred organometallic compounds of silver are silver cyanide [AgCN] and silver acetyl acetonate [Ag(acac)].

Any organometallic compound of copper comprising at least one C—Cu bond is suitable for carrying out said step b1). Advantageously, copper cyanide [CuCN], copper acetyl acetonate [Cu(acac)], (N,N′-diisopropylacetamidinato) copper [(Cu(iPr-Me-AMD))₂], N,N′-diisopropyl-2-n-butylamidinato copper [(Cu(iPr-nBu-AMD))₂], copper hexafluoro-acetylacetonato [(Cu(hfac)₂], cyclopentadienyl copper triethyl phosphine, cyclopentadienyl copper tertiary-butylisocyanide, copper ethylhexanoate [Cu(OOCCH(C₂H₅)C₄H₉)₂], copper bis(2,2,6,6-tetramethyl-3,5-heptanedionate) [Cu(OCC(CH₃)₃CHCOC(CH₃)₃)₂], are used. Preferred organometallic copper compounds are copper cyanide [CuCN] and copper acetyl acetonate [Cu(acac)].

All of the organometallic compounds cited above and their preparation methods are known to the skilled person. Said compounds are either commercially available or described in the literature.

In accordance with a first particular implementation of the preparation process of the invention, step a1) is followed by a step a2), prior to carrying out said step b1), said step a2) consisting of reducing said monometallic catalyst obtained at the end of said step a1) in the presence of at least one reducing gas, preferably hydrogen. More precisely, said step a2) generally consists of a slow ramp-up of temperature, for example in the range 0.1° C./minute to 5° C./minute, in a stream of reducing gas, preferably in hydrogen, to a maximum reduction temperature in the range 100° C. to 600° C., preferably in the range 200° C. to 500° C., followed by maintaining said temperature for a period in the range 1 hour to 40 hours, preferably in the range 5 hours to 30 hours. Said conditions mean that a supported monometallic catalyst which is very substantially reduced can be obtained.

In accordance with a second particular implementation of the preparation process of the invention, said step b1) is followed by at least one step c1) during which said supported catalyst containing said metallic phase based on nickel and at least one metal from group IB obtained at the end of said step b1) undergoes at least one drying phase. Said step c1) is carried out under vacuum, in a stream of inert gas (nitrogen, argon, helium) or air, preferably under vacuum or in a stream of inert gas. It is carried out at a temperature in the range 10° C. to 150° C., preferably in the range 20° C. to 50° C.

In accordance with a particular third implementation of the preparation process of the invention, said step b1) is followed by at least one step c2) during which said supported catalyst containing said metallic phase based on nickel and at least one metal from group IB obtained at the end of said step b1) undergoes at least one washing phase. The wash(es) is(are) preferably carried out using a hydrocarbon, for example that which may have been used as a solvent in said step b1) when it is carried out in the liquid phase.

In accordance with the preparation process of the invention, said second and third particular implementational modes described above are independent of each other. Advantageously, said preparation process of the invention employs at least said step c1) and at least said step c2), said steps c1) and c2) being carried out following said step b1) in any order. Preferably, the catalyst from said step b1) undergoes at least one drying step in accordance with c1) then at least one washing step in accordance with c2). A new drying step in accordance with c1) is also often carried out following a washing step in accordance with c2).

In accordance with a fourth particular implementation of the preparation process of the invention, said step b1) is followed by a step d1) consisting of carrying out activation of the catalyst obtained at the end of said step b1) in the presence of at least one reducing gas, preferably hydrogen. Said step d1) is carried out at a temperature in the range 150° C. to 600° C., preferably in the range 200° C. to 500° C., more preferably in the range 300° C. to 500° C., for a period in the range 1 minute to 30 hours, preferably in the range 10 minutes to 10 hours. The ramp-up to this temperature of activation is generally slow, for example in the range 0.1° C./minute to 5° C./minute. This activation in the presence of a reducing gas may be carried out either in a static manner or in a stream of reducing gas, preferably in a stream of reducing gas.

In accordance with the preparation process of the invention, one or more particular implementations described above may be carried out when implementing said process. Preferably, steps a1), b1), c1), c2) and d1) are carried out in succession and still more preferably, steps a1), a2), b1), c1), c2) and d1) are carried out in succession.

The catalyst obtained after carrying out said step b1) or after carrying out said step c1) and/or said step c2) or after carrying out said step d1) may be used directly in a reaction unit carrying out the conversion of a hydrocarbon feed, in particular in a reaction unit carrying out the selective hydrogenation of a polyunsaturated hydrocarbon feed. Said catalyst prepared using the process of the invention may also be stored in air then reduced prior to use. The reduction then generally consists of a slow temperature ramp-up, for example in the range 0.1° C./minute to 5° C./minute, in a stream of reducing gas, preferably in hydrogen, to the maximum reduction temperature, in the range 100° C. to 600° C., preferably in the range 200° C. to 500° C., followed by maintaining that temperature for a period in the range 1 hour to 40 hours, preferably in the range 5 hours to 30 hours.

The present invention also pertains to a process for the selective hydrogenation of a polyunsaturated hydrocarbon feed, said process comprising passing said feed into at least one reaction unit provided with at least one supported catalyst comprising at least one metallic phase containing nickel and at least one metal M from group IB prepared in accordance with the preparation process of the invention.

Said polyunsaturated hydrocarbon feed treated in the selective hydrogenation process of the invention is advantageously a steam cracked gasoline comprising polyunsaturated hydrocarbons containing at least 4 carbon atoms and having an end point of up to 220° C. More precisely, said polyunsaturated hydrocarbons present in the feed treated using the selective hydrogenation process of the invention are in particular diolefin compounds, styrene compounds and indene compounds. Regarding the diolefin compounds, said feed in particular contains butadiene, isoprene and cyclopentadiene. Regarding the styrene compounds, said feed in particular contains styrene and alpha-methyl styrene. Regarding the indene compounds, said feed in particular contains indene.

The selective hydrogenation process of the invention is intended to selectively hydrogenate said polyunsaturated hydrocarbons present in said feed to be treated in a manner such that the diolefin compounds are partially hydrogenated into mono-olefins and such that the styrene and indene compounds are partially hydrogenated into the corresponding aromatic compounds.

The effluent obtained after carrying out the selective hydrogenation process of the invention has a substantially reduced polyunsaturated hydrocarbons content; in particular, it has a reduced diolefin compounds, styrene compounds and indene compounds content, while retaining a quantity of aromatic compounds (more precisely a quantity of aromatic rings) close to that present in said hydrocarbon feed. Said effluent is advantageously upgradeable as a base in a gasoline or can be used as a base for upgrading aromatic compounds.

The selective hydrogenation process of the invention is advantageously carried out under pressure, in the liquid phase, in the presence of a quantity of hydrogen that is in slight excess with respect to the stoichiometric value allowing the selective hydrogenation of the polyunsaturated compounds present in the hydrocarbon feed, i.e. an excess which is generally in the range 5% to 30%. The selective hydrogenation process of the invention is carried out at a temperature in the range 20° C. to 200° C. The pressure is generally sufficient to maintain at least 80% by weight of the feed to be treated in the liquid phase at the inlet to the reaction unit. It is generally in the range 0.4 MPa to 5 MPa, more advantageously in the range 1 MPa to 4 MPa. The hourly space velocity (defined as the ratio of the volume flow rate of feed to the volume of catalyst) established under these conditions is generally in the range 0.2 h⁻¹ to 30 h⁻¹, and preferably in the range 1 h⁻¹ to 20 h⁻¹, more preferably in the range 2 h⁻¹ to 10 h⁻¹.

The selective hydrogenation process is technically undertaken, for example, by injection, as an upflow or downflow, of the feed and hydrogen into a fixed bed reactor. It may also advantageously be carried out by implanting at least said supported catalyst containing nickel and at least one metal from group IB in a reactive distillation column or in reactor-exchangers.

The following examples illustrate the invention without limiting its scope.

### Example 1

### (Comparative): Preparation of a Monometallic Catalyst A Supported on Silica and Containing Nickel

A Ni/silica (catalyst A) type catalyst was prepared by cation exchange of the [Ni(NH₃)₆]²⁺ salt on silica. The impregnation solution was prepared by mixing a solution of nickel nitrate in a concentration of 7×10⁻³ mol/L with an ammoniacal solution in a concentration of 0.4 mol/L. A quantity of 4 g of silica (Aerosil-200, Degussa) was brought into contact with the impregnation solution for 24 hours, at ambient temperature and with stirring. The solid was then recovered by filtering, washed with permutated water, dried in a vacuum oven at 80° C. then 100° C. and finally treated in a stream of hydrogen at 500° C. before being cooled and stored in air.

The monometallic catalyst A obtained thereby contained 11.7% by weight of Ni (according to elemental analysis).

### Example 2

### (In Accordance with the Invention): Preparation of a Catalyst B Supported on Silica and Containing Nickel and Gold

A quantity of 40 mg of monometallic catalyst A was pre-reduced in a stream of hydrogen at 400° C. for 14 hours. A catalyst B was prepared by bringing 40 mg pre-reduced monometallic catalyst A into contact with a suspension of gold cyanide (AuCN, Strem) in heptane (Acros) (4 mg of AuCN in 20 mL of n-heptane) at ambient temperature, in the presence of hydrogen and with stirring (magnetic stirrer, magnetic bar). After 12 hours of contact, catalyst B obtained thereby was introduced directly into the autoclave for the test for the selective hydrogenation of styrene illustrated in Example 6.

### Example 3

### (In Accordance with the Invention): Preparation of a Catalyst C Supported on Silica and Containing Nickel and Gold

A quantity of 1.08 g of monometallic catalyst A was pre-reduced in a stream of hydrogen at 400° C. for 14 hours. A catalyst C was prepared by bringing 1.08 g pre-reduced monometallic catalyst A into contact with a suspension of gold cyanide (AuCN, Strem) in heptane (Acros) (100 mg of AuCN in 20 mL of n-heptane) at ambient temperature, in the presence of hydrogen and with stirring (magnetic stirrer, magnetic bar). After 12 hours of contact, the solid was vacuum dried (10⁻¹ mbar), washed 4 times with 20 mL of n-heptane and placed under vacuum (10⁻¹ mbar) again at ambient temperature. It was then stored under argon (1 atm). Catalyst C obtained thereby contained 11.5% by weight of nickel and 9.6% by weight of gold (according to elemental analysis), corresponding to an Au/Ni molar ratio of 0.25.

### Example 4

### (In Accordance with the Invention): Preparation of a Catalyst D Supported on Silica and Containing Nickel and Gold

A quantity of 1.08 g of monometallic catalyst A was pre-reduced in a stream of hydrogen at 400° C. for 14 hours. A catalyst D was prepared by bringing 1.08 g pre-reduced monometallic catalyst A into contact with a suspension of gold cyanide (AuCN, Strem) in heptane (Acros) (100 mg of AuCN in 20 mL of n-heptane) at ambient temperature, in the presence of hydrogen and with stirring (magnetic stirrer, magnetic bar). After 12 hours of contact, the solid was vacuum dried (10⁻¹ mbar), washed 4 times with 20 mL of n-heptane and placed under vacuum (10⁻¹ mbar) again at ambient temperature, then stored under argon (1 atm). It was then brought into the presence of hydrogen (100 mbar) and heated to 400° C. for 2 hours (temperature ramp-up of 1.5° C./minute). Finally, it was stored under argon at ambient temperature.

Catalyst D obtained thereby contained 11.5% by weight of nickel and 9.6% by weight of gold (according to elemental analysis), corresponding to an Au/Ni molar ratio of 0.25.

### Example 5

### (Comparative): Preparation of a Catalyst E Supported on Silica and Containing Nickel and Gold Using a Metallic Gold Salt as the Precursor

An aqueous solution of the salt [Au(NH₃)₄]³⁺ was pre-prepared by dissolving 31 mg of Au(NH₃)₄(NO₃)₃ in 20 mL of distilled water. The complex Au(NH₃)₄(NO₃)₃ was itself pre-prepared by dissolving chloroauric acid HAuCl₄ (Aldrich) in an aqueous solution of ammonium nitrate (Aldrich); concentrated ammonia was added drop by drop so that the pH was always below 5. The complex formed thereby was washed with water and ether, vacuum dried then stored in air.

The monometallic catalyst A was pre-reduced in a stream of hydrogen at 400° C. for 14 hours. A catalyst E was prepared by bringing 0.5 g of pre-reduced catalyst A into contact with 20 mL of an aqueous solution of the salt {Au(NH₃)₄}³⁺ for 5 hours, at ambient temperature, in the presence of hydrogen and with stirring. The solid was then recovered by filtering, washed with ethanol then with heptane, dried under vacuum then in air at ambient temperature and then treated in a stream of hydrogen at 400° C.

Catalyst E obtained thereby contained 11.6% by weight of nickel and 2.6% by weight of gold (according to elemental analysis).

### Example 6

### Catalytic Performances of Catalysts A to E in Styrene Hydrogenation

The catalytic properties of the catalysts prepared in the above examples were evaluated successively in a styrene hydrogenation process. The selective hydrogenation of styrene produces ethylbenzene, this hydrogenation constituting the desired reaction. The complete hydrogenation of styrene produces ethylcyclohexane, which is produced by an unwanted, successive reaction.

Styrene was hydrogenated in a 100 mL stainless steel autoclave provided with mechanical, magnetically actuated stirring and which could function at a maximum pressure of 100 bar and at temperatures in the range 5° C. to 200° C.

A quantity of catalyst (40 mg) was transferred into the autoclave in the absence of air. The monometallic catalyst A was pre-reduced in a stream of hydrogen at 400° C. for 14 hours. The other catalysts B, C, D and E were used as they were. After adding 50 mL of n-heptane, the autoclave was sealed, purged then pressurized to 15 bar (0.15 MPa) of hydrogen. After introducing styrene, it was heated at 150° C. for 2 hours with stirring then brought back to the initial test temperature (5° C.). At time t=0, 3.5 g of styrene and an internal reference were introduced into the autoclave and stirring was commenced (500 rpm). The progress of the reaction was monitored by taking samples from the reaction medium at regular time intervals. These samples were analyzed by gas chromatography. Once all of the styrene had been consumed, the pressure in the autoclave was raised to 60 bar and the temperature was raised rapidly to 150° C. (in less than 1 hour): in this second step, the reaction that took place was the hydrogenation of ethylbenzene to ethylcyclohexane.

The performances of the catalysts were evaluated in terms of selectivity, which was evaluated from a measurement of the reaction rates for the hydrogenation reactions. The reaction rates for the two reactions of hydrogenation of styrene to ethylbenzene and hydrogenation of ethylbenzene to ethylcyclohexane were defined as the slopes at the origin with respect to the mass of catalyst in the graphs of the change of ethylbenzene concentration with time. The reaction rate r1 for the hydrogenation of styrene to ethylbenzene was determined in the first step of the test. The reaction rate r2 for the hydrogenation of ethylbenzene to ethylcyclohexane was determined in the second step of the test. The selectivity of the catalyst for the desired product, namely ethylbenzene, was evaluated by the ratio r1/r2; the higher the ratio, the more selective is the catalyst for ethylbenzene.

In Table 1, the selectivities of the catalysts prepared in accordance with the above examples are compared with that of the monometallic Ni catalyst (catalyst A)

TABLE 1
Selectivities for catalysts supported on silica. Comparison with
monometallic catalyst A based on nickel alone

| | Selectivity |
|---|---|
| Catalyst | (r1/r2)/(r1_{ref}/r2_{ref}) |
| A | 1 |
| B | 3.2 |
| C | 3.1 |
| D | 5.9 |
| E | 1.0 |

In Table 1, r1_{ref} and r2_{ref} respectively correspond to the reaction rate r1 defined above, or respectively r2 defined above, each being measured using the corresponding monometallic catalyst A.

Catalysts B, C and D based on nickel and gold prepared using the process of the invention have a selectivity 3 to 6 times higher than that of the monometallic nickel catalyst (catalyst A). For these catalysts B, C and D based on nickel and gold, the rate of hydrogenation of ethylbenzene to ethylcyclohexane was thus greatly slowed compared with the rate of hydrogenation of styrene to ethylbenzene. For said catalysts B, C and D, hydrogenation of the aromatic ring was thus substantially reduced compared with that observed with catalyst A, which latter favoured the production of ethylcyclohexane to the detriment of ethylbenzene. Thus, catalysts B, C and D are much more selective for ethylbenzene than catalyst A. Adding gold can thus slow down the reaction for the hydrogenation of the aromatic ring of styrene.

Catalysts C and D prepared using an identical protocol with the exception of the final activation treatment (in the presence of hydrogen, at 400° C., for 2 hours) undergone only by catalyst D, illustrates the importance of this activation treatment. It allows the selectivity of the catalyst as regards ethylbenzene to be significantly increased.

Catalysts B, C and D prepared using the process of the invention are also much more selective than catalyst E which was prepared in the presence of a metallic gold salt in an aqueous medium.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosures of all applications, patents and publications, cited herein and of corresponding French application Ser. No. 09/03.987, filed Aug. 17, 2009, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A process for preparing a catalyst comprising at least one porous support and at least one metallic phase containing nickel and at least one metal M from group IB in a proportion such that the molar ratio M/Ni is in the range of 0.005 to 0.5, said process comprising at least the following steps in succession:
a1) depositing nickel on at least said support in order to obtain a supported nickel-based monometallic catalyst; and
b1) depositing, in the presence of at least one reducing gas and in the absence of any aqueous solvent, at least one organometallic compound of at least said metal M onto said monometallic catalyst to provide a molar ratio M/Ni in the range of 0.005 to 0.5.

2. A preparation process according to claim 1, in which said catalyst has a M/Ni molar ratio in the range of 0.01 to 0.5.

3. A preparation process according to claim 1, in which said porous support present in said catalyst is an alumina or a silica.

4. A preparation process according to claim 1, in which said metal M from group IB is gold.

5. A preparation process according to claim 1, in which said step b1) is carried out in the gas phase.

6. A preparation process according to claim 1, in which said step b1) is carried out in the liquid phase in an organic solvent.

7. A preparation process according to claim 1, in which said organometallic compound is gold cyanide when said metal M is gold.

8. A preparation process according to claim 1, in which said step a1) is followed by a step a2) prior to carrying out said step b1), said step a2) comprising carrying out a reduction of said monometallic catalyst in the presence of at least one reducing gas.

9. A preparation process according to claim 1, in which said step b1) is followed by at least one step c1) during which said supported catalyst obtained at the end of said step b1) undergoes at least one drying phase.

10. A preparation process according to claim 1, in which said step b1) is followed by at least one step c2) during which said supported catalyst obtained at the end of said step b1) undergoes at least one washing phase.

11. A preparation process according to claim 1, in which said step b1) is followed by a step d1) comprising activating the catalyst obtained at the end of said step b1) in the presence of at least one reducing gas.

12. A process for the selective hydrogenation of a feed of polyunsaturated hydrocarbons, comprising passing said feed into at least one reaction unit provided with at least one supported catalyst comprising at least one metallic phase prepared in accordance with the preparation process according to claim 1.

13. A selective hydrogenation process according to claim 12, in which said feed of polyunsaturated hydrocarbons is a steam cracked gasoline comprising polyunsaturated hydrocarbons containing at least 4 carbon atoms and having an end point of up to 220° C.

14. A selective hydrogenation process according to claim 13, in which said polyunsaturated hydrocarbons are diolefin compounds, styrene compounds and indene compounds.

15. A selective hydrogenation process according to claim 12, carried out at a temperature in the range 20° C. to 200° C., at a pressure in the range 0.4 MPa to 5 MPa, at an hourly space velocity (defined as the ratio of the volume flow rate of feed to the volume of catalyst) in the range 0.2 h⁻¹ to 30 h⁻¹.

16. A preparation process according to claim 4, in which said catalyst has a M/Ni molar ratio in the range of 0.01 to 0.5.

17. A preparation process according to claim 1, in which said catalyst has a M/Ni molar ratio in the range of 0.03 to 0.3.

18. A preparation process according to claim 4, in which said catalyst has a M/Ni molar ratio in the range of 0.03 to 0.3.

19. A preparation process according to claim 1, wherein the Au/Ni ratio is at least about 0.25.
